(19) United States
(12) Patent Application Publication
(10) Pub. No.: US 2012/0264949 A1
(43) Pub. Date: Oct. 18, 2012

(54) Method of Labeling Dopamine D2 Receptor Using Radiosynthesized Ligand of Iodine-123-Epidepride

(75) Inventors: Shih-Ying Lee, Taoyuan County (TW); Kang-Wei Chang, Taoyuan County (TW); Chia-Chieh Chen, Taoyuan County (TW)

(73) Assignee: ATOMIC ENERGY COUNCIL-INSTITUTE OF NUCLEAR ENERGY RESEARCH, Taoyuan County (TW)

(21) Appl. No.: 13/086,254

(22) Filed: Apr. 13, 2011

(51) Int. Cl. C07D 207/09

(52) U.S. Cl. 548/567

(57) ABSTRACT

A method is provided to label dopamine D₂ receptors at striatum and areas outside of striatum. A radiosynthesized ligand of iodine(I)-123-Epidepride is used. The I-123-Epidepride can be strongly bonded to the D₂ receptor and has a rare characteristic of non-specificity. Hence, it is suitable for developing a tracer for areas outside of striatum, where D₂ receptor densities are low. Besides, I-123-Epidepride can be passed through blood brain barrier and has a high affinity to an animal's brain, so it can be used to develop medicines for diagnosing schizophrenia.

### TECHNICAL FIELD OF THE DISCLOSURE

The present disclosure relates to labeling dopamine D₂ receptors; more particularly, relates to providing an iodine(I)-123 probe of dopamine D₂ receptors for striatum and areas outside of striatum to be used for making an I-123 medicine for diagnosing schizophrenia.

### DESCRIPTION OF THE RELATED ARTS

Productions of medicines for schizophrenia are growing today and schizophrenia is obviously becoming a serious disease for modern people. For diagnosing schizophrenia, two out of five syndromes have to be found in a patient and the syndromes found have to last for at least 1 month. The five syndromes include delusions, hallucinations, disorganized speech, negative symptoms of schizophrenia and catatonia. The first two are positive syndromes and the last three are negative syndromes.

Schizophrenia can be inherited at a higher ratio of about 80%. Presuppositions for schizophrenia suggest that the disease may be related to the dopamine system:

(1) Mesolimbic pathway: When the density of dopamine under the cortex gets high, dopamine D₂ receptors will be over-excited and activated and positive syndromes may happen.

(2) Mesocortical pathway: When the density of dopamine in prefrontal lobes becomes low, dopamine D₂ receptors will be under-excited and cognitive defects may be caused with negative syndromes.

(3) Corticallimbic pathway: When the corticallimbic pathway is low in function, the mesolimbic pathway cannot be restrained and positive syndromes may thus happen.

It has been confirmed in many studies that, in areas outside of striatum (like prefrontal cortex and temporal lobe), changes in structure and function may be found in a schizophrenia patient. These studies include “Local and Distributed Effects of Apomorphine on Fronto-Temporal Function in Acute Unmedicated Schizophrenia” by Fletcher PC, etc. (J Neurosci, 1996, Vol. 16, pp. 7055-7062), “Disrupted Pattern of D₂ Dopamine Receptors in the Temporal Lobe in Schizophrenia” by Goldsmith SK, etc. (Arch Gen Psychiatry, 997, Vol. 54, pp. 649-658) and “Lamina-Specific Alterations in the Dopamine Innervation of the Prefrontal Cortex in Schizophrenic Subjects” by Akil M, etc. (Am J Psychiatry, 999, Vol. 156, pp. 1580-1589). Besides, in “Dopamine D₂/3 Receptor Binding Potential and Occupancy in Midbrain and Temporal Cortex by Haloperidol, Olanzapine and Clozapine” by Tuppurainen H, etc. (Eur Arch Psychiatry Clin Neurosci, 2006, Vol. 256(6), pp. 382-387), it is confirmed that in a schizophrenia patient's brain, dopamine D₂ receptors in the midbrain area have a lower density than that in a normal person's brain.

Epidepride was fabricated by Clanton JA, etc. early in 1991 (“Preparation of [123I]- and [125I]Epidepride: A Dopamine D-2 Receptor Antagonist Radioligand”, J Labelled Compd Radiopharm, 1991, Vol. 29, pp. 745-751), which is an imaging agent for dopamine D₂ receptors. Epidepride is similar to FLB457 [51]. FLB457 [51] is mainly used in positron emission tomography (PET) while Epidepride is used in single photon emission computed tomography (SPECT), as shown in FIG. 10 and FIG. 3. Imaging agents found in the market only include Raclopride [52] and IBZM [53], as shown in FIG. 11 and FIG. 12.

In areas outside of striatum, the density of dopamine D₂ receptors is only 1/10 to 1/100 to that in striatum. If IBZM (whose KD value is 0.43 nM) is used as the imaging agent, it is obvious that the image of dopamine D₂ receptors in areas outside of striatum is hard to be obtained. Hence, the prior arts do not fulfill all users' requests on actual use.

### SUMMARY OF THE DISCLOSURE

The present disclosure is to provide an I-123 probe of dopamine D₂ receptors for striatum and areas outside of striatum to be used for making an I-123 medicine for diagnosing schizophrenia.

To achieve the above purpose, the present disclosure is a method of labeling dopamine D₂ receptors using a radiosynthesized ligand of I-123-Epidepride, comprising steps of: (a) obtaining a precursor of Sn-Epidepride to be added with methanol to be oscillated for obtaining a mixed solution of Sn-Epidepride; (b) mixing the mixed solution of Sn-Epidepride with a solution of I-123-ammonium iodide (NH₄I) to be filled with a solution of hydrogen peroxide to be oscillated and then to be stayed still for processing destannylation; (c) filling the mixed solution of Sn-Epidepride with a solution of sodium bisulfite to stop destannylation and, after destannylation, adding the mixed solution of Sn-Epidepride with a saturated buffer solution of disodium hydrogen phosphate to be neutralized; (d) filling the mixed solution of Sn-Epidepride into a column, washing out un-reacted I-123 ions from the column with sterile water and eluting the column with 100% dehydrated alcohol to obtain a product of I-123-Epidepride having a radiochemistry purity higher than 90%; and (e) filtering out the product of I-123-Epidepride through a filtering cartridge to be stored in a sterile glass bottle. Accordingly, a novel method of labeling dopamine D₂ receptors using a radiosynthesized ligand of I-123-Epidepride is obtained.

### BRIEF DESCRIPTIONS OF THE DRAWINGS

The present disclosure will be better understood from the following detailed description of the preferred embodiment according to the present disclosure, taken in conjunction with the accompanying drawings, in which

FIG. 1 is the flow view showing the preferred embodiment according to the present disclosure.

FIG. 2 is the block view showing the preferred embodiment.

FIG. 3 is the view showing the chemical reaction for making I-123-Epidepride.

FIG. 4 is the view showing the radiochemistry purity of I-123-Epidepride obtained by using Radio-TLC.

FIG. 5 is the view showing the radiochemistry purity of I-123-Epidepride obtained by using HPLC.

FIG. 6 is the view showing the lipophilicity.

FIG. 7 is the view showing the labeling results.

FIG. 8A is the view showing the stability at 0 hour.

FIG. 8B is the view showing the stability at 24 hour.

FIG. 9 is the view showing the combined image of a mouse brain obtained through microSPECT and microCT.

FIG. 10 is the structural view of FLB457.

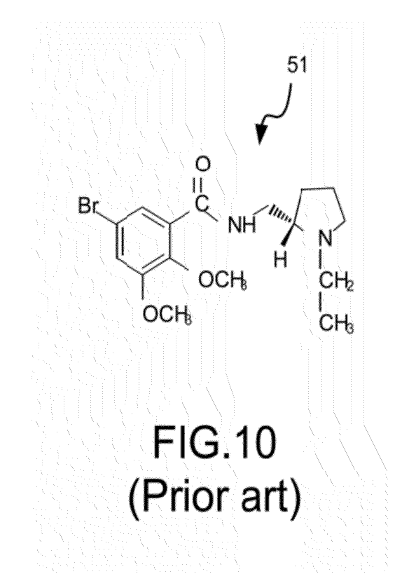

FIG. 11 is the structural view of Raclopride.

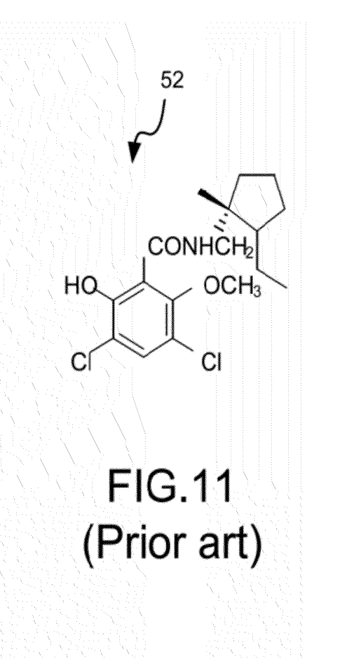

FIG. 12 is the structural view of IBZM.

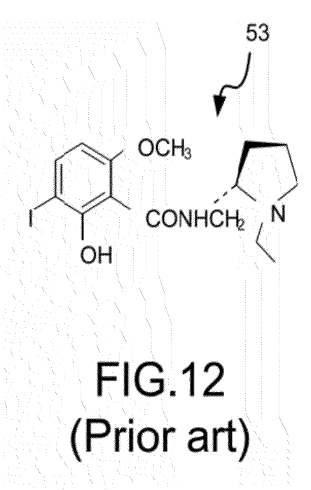

### DESCRIPTION OF THE PREFERRED EMBODIMENT

The following description of the preferred embodiment is provided to understand the features and the structures of the present disclosure.

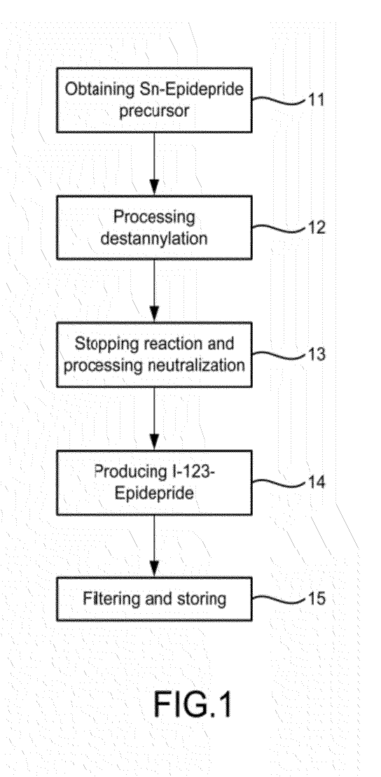

FIG. 1 is a flow view showing a preferred embodiment according to the present disclosure. As shown in the figure, the present disclosure is a method of labeling dopamine D₂ receptors using a radiosynthesized ligand of iodine(I)-123-Epidepride, where an I-123 probe is provided for labeling dopamine D₂ receptors at striatum and areas outside of striatum. The present disclosure comprises the following steps:

(a) Obtaining Sn-Epidepride precursor [1,1]: A precursor of Sn-Epidepride is obtained to be added into methanol, where the Sn-Epidepride precursor has an amount of 150˜250 micro-gram (μg) and the methanol has an amount of 50˜150 micro-liter (μl). After being oscillated, a mixed solution of Sn-Epidepride is obtained.

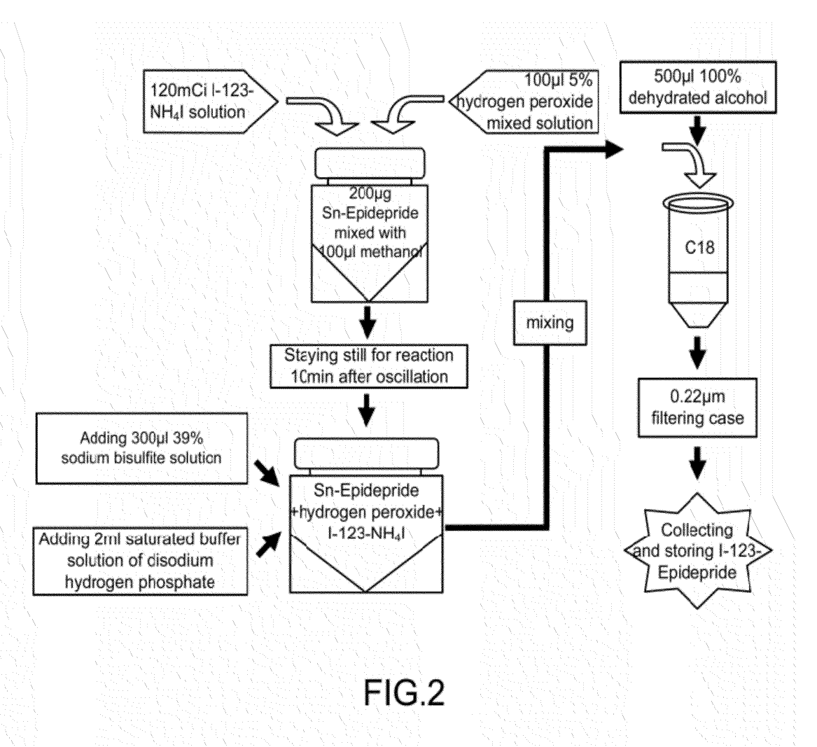

(b) Processing destannylation [12]: The mixed solution of Sn-Epidepride is mixed with a solution of I-123-ammonium iodide(NH₄I), where the solution of I-123-NH₄I has an amount of 200˜300 μl. Then, the mixed solution of Sn-Epidepride is filled with a solution of hydrogen peroxide to be oscillated and then is stayed still to process destannylation, where the solution of hydrogen peroxide has an amount of 50˜150 μl. In FIG. 2, an original radioactivity of I-123-NH₄I is 100˜200 millicuries (mCi).

(c) Stopping reaction and processing neutralization [13]: The mixed solution of Sn-Epidepride is filled with a solution of 39% sodium bisulfite to stop destannylation, where the solution of sodium bisulfite has an amount of 250˜350 μl. After destannylation, the mixed solution of Sn-Epidepride is added with a saturated buffer solution of disodium hydrogen phosphate to be neutralized, where the buffer solution of disodium hydrogen phosphate has an amount of 1˜3 milliliter (ml).

(d) Producing I-123-Epidepride [14]: The mixed solution of Sn-Epidepride is filled into a C18 column for washing out un-reacted I-123 ions by sterile water. Then, the C18 column is eluted with 100% dehydrated alcohol to obtain a product of I-123-Epidepride, where the dehydrated alcohol has an amount of 450˜550 μl and the product of I-123-Epidepride has a radiochemistry purity higher than 90%.

(e) Filtering and storing [15]: The product of I-123-Epidepride is filtered out through a filtering cartridge to be stored in a sterile glass bottle. On using the product of I-123-Epidepride, the product is diluted with a sterile saline solution to obtain a final solution having an ethanol density smaller than 20%.

The Sn-Epidepride precursor is a white powder with a purity higher than 99% and has a full name as (S)-5-(tri-n-butyltin)-N-((1-ethyl-2-pyrrolidinyl)methyl)-2,3-d imethoxybenzamide, whose chemical formula is C₂₈H₅₀N₂O₃Sn having a molecular weight of 581.42.

The solution of hydrogen peroxide is obtained by filling a bottle of 350 μl of sterile water with 100 μl of 30% hydrogen peroxide; then, 150 μl of glacial acetic acid is added to obtain a solution of 5% hydrogen peroxide. The C18 column is washed back and forth for 5 times by 3 ml of 100% dehydrated alcohol and 5 ml of sterile water, and then is put on a device of leaded glass. The saturated buffer solution of disodium hydrogen phosphate is obtained with a powder of disodium hydrogen phosphate.

The 30% hydrogen peroxide, the glacial acetic acid, the 100% dehydrated alcohol, the 39% sodium bisulfite, the methanol and the powder of disodium hydrogen phosphate are American Chemical Society (ACS) level medicines. The I-123-NH₄I is obtained through an irradiation of Xe-123 gas target by a proton beam of compact cyclotron, and the I-123-NH₄I used in the present disclosure has to have an original radioactivity higher than 100 mCi and an amount smaller than 300 μl for a good yield.

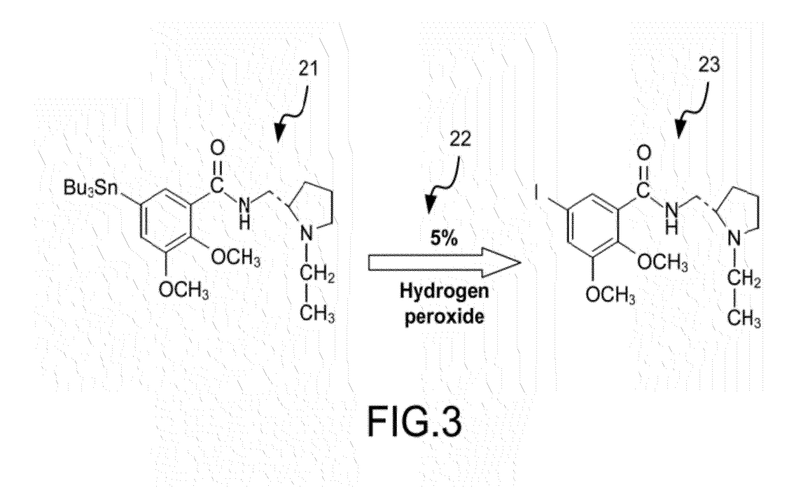

FIG. 2 and FIG. 3 are a block view showing the preferred embodiment and a view showing a chemical reaction for making I-123-Epidepride. As shown in the figures, on processing labeling, 200 μg/vial of Sn-Epidepride precursor is obtained to be added into 100 μl of methanol; then, the mixed solution is oscillated for fully dissolving the Sn-Epidepride precursor. Then, 300 μl of an I-123-NH₄I solution, which has an activity of 120 mCi, is put into a V-shaped reaction bottle. Then, a 100 μl solution with Sn-Epidepride dissolved is filled in and is added with 100 μl of 5% hydrogen peroxide to be oscillated for mixing evenly. Then, the mixed solution is stayed still for 10 minutes (min) for processing destannylation. In FIG. 3, for making the I-123-Epidepride [23], a Sn-Epidepride precursor [21] and I-123-NH₄I are obtained for processing destannylation through an oxidation of a solution of 5% hydrogen peroxide [22]. In an acidic environment, tributyl tin on the Sn-Epidepride precursor is dropped off while NH₄I is oxidized by hydrogen peroxide with iodine ions released as a covalent bond at the original position for tributyl tin.

Thereafter, oscillation is processed for 10 min and then 300 μl of a solution of 39% sodium bisulfite is added into the bottle to stop the reaction for preventing over-reaction, while 2 ml of a saturated buffer solution of disodium hydrogen phosphate is added for neutralization. After the reaction stops, the reacted solution of Sn-Epidepride is taken out to be filled into a C18 column for washing out un-reacted I-123 ions from the C18 column by 5 ml of sterile water. Then, the C18 column is eluted with 500 μl of 100% dehydrated alcohol for obtaining a product of I-123-Epidepride. At last, the product of I-123-Epidepride is filtered out through a filtering cartridge for removing impurities and bacteria, where the filtering cartridge has 0.22 μm openings. Therein, on using the present disclosure, the product of I-123-Epidepride is diluted with a sterile saline solution to obtain a final solution having an ethanol density smaller than 20%.

On examining a radiochemistry purity of the I-123-Epidepride, Radio-TLC or HPLC is used, where the radiochemistry purity of the I-123-Epidepride have to be higher than 90%.

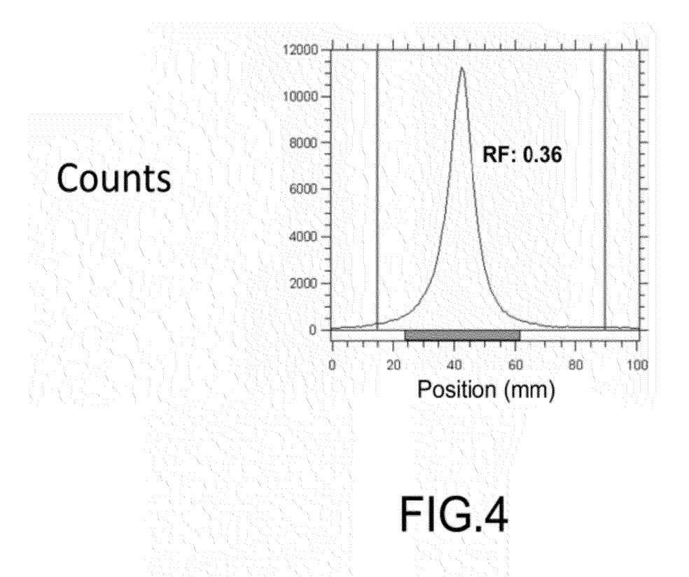

FIG. 4 is a view showing a radiochemistry purity of I-123-Epidepride obtained by using Radio-TLC. As shown in the figure, Radio-TLC is used for analyzing a radiochemistry purity of I-123-Epidepride. The analysis uses a 1.5×13 cm ITLC-SG in a solid phase; and a condition solvent is obtained by mixing chloroform and methanol at a ratio of 9:1. As a result, a radio frequency (RF) of the I-123-Epidepride is 0.36 with a radiochemistry purity greater than 90%.

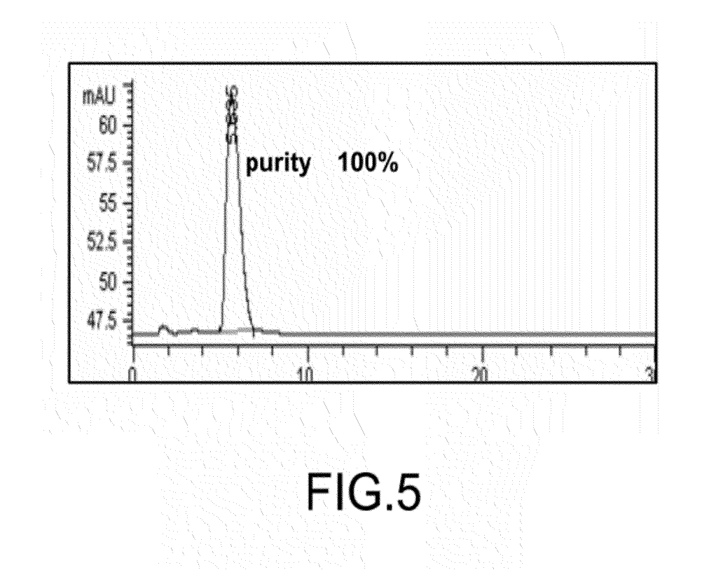

FIG. 5 is a view showing a radiochemistry purity of I-123-Epidepride obtained by using HPLC. As shown in the figure, a radio detector for HPLC is used for analyzing a radiochemistry purity of I-123-Epidepride. A C18 column is used, which is a column for HPLC and has a size of 3.9×150 mm. The analysis uses an elute buffer obtained by mixing acetonitrile and 0.01M phosphoric acid at a ratio of 30:70. The elute buffer is flowed at a speed of 1 mL/min and the wavelength for analysis is 230 nanpmeters (nm). As a result, a staying period for the I-123-Epidepride is 5.635 min with a radiochemistry purity greater than 90%.

For analyzing lipophilicity of the I-123-Epidepride, a solution of phosphate buffer saline (PBS) is used with lipophilic octanol. After 50 μl of I-123-Epidepride, 0.5 ml of PBS and 0.5 ml of octanol are mixed, a diluted octanol solution is obtained with an aqueous solution which has an equal weight as the octanol solution and then is processed with a gamma-counter. Therein, the lipophilicity of the I-123-Epidepride is expressed as a value of log P and log P is obtained by the following formula: Log P=Log {(Decay corrected activity)organic layer×10/(Decay corrected activity)aqueous layer}.

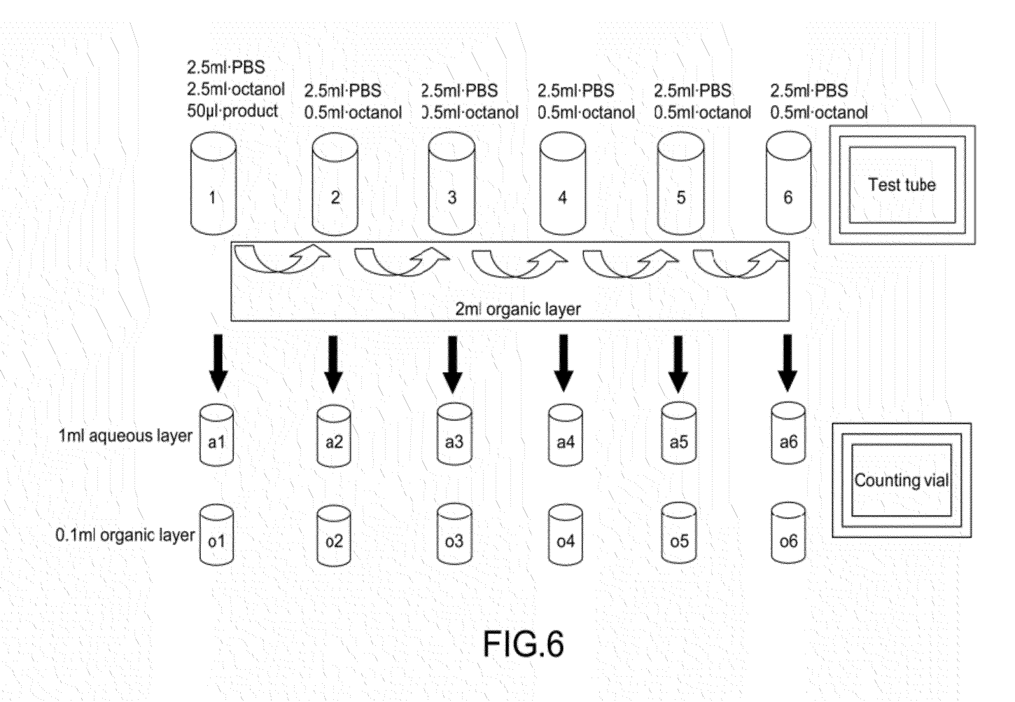

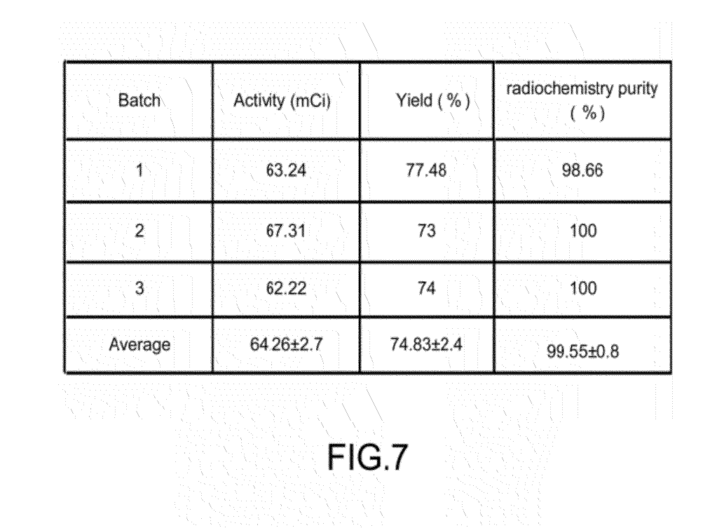

FIG. 6 and FIG. 7 are views showing lipophilicity and labeling results. As shown in the figures, after calculation, the log P value of the I-123-Epidepride is 1.76±0.04 (shown in FIG. 6), which means a high lipophilicity for breaking blood brain barrier (BBB). Besides, the labeling method according to the present disclosure obtains 20 mCi each time for three times with an average radiochemistry purity of 99.55±0.8% and an average yield of 74.83±2.4% (shown in FIG. 7).

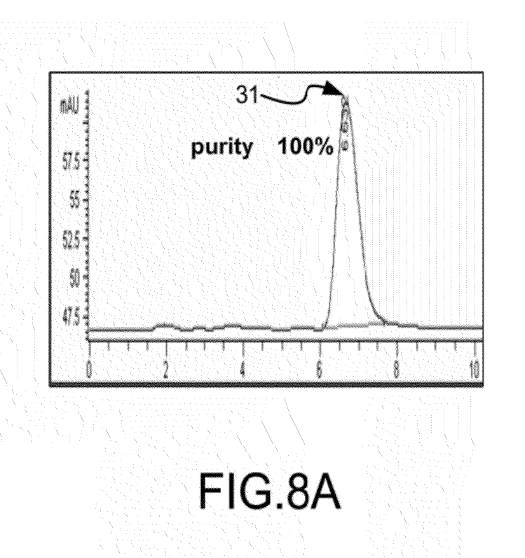

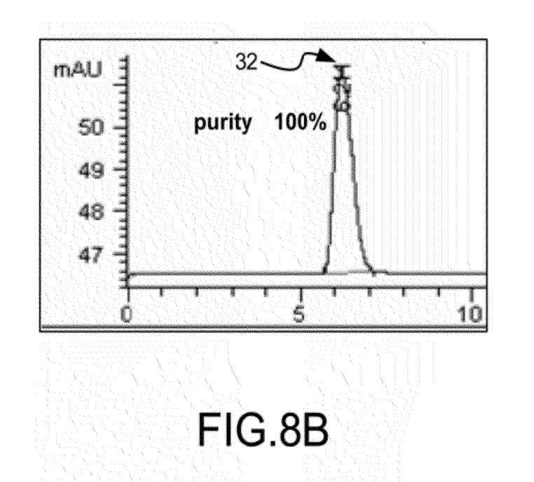

FIG. 8A and FIG. 8B are views showing stability at 0 hour and 24 hour. As shown in the figures, after the I-123-Epidepride is stayed still for 24 hours, its radiochemistry purity is measured. As shown by the liquid peaks [31], [32], the stability is higher than 90%.

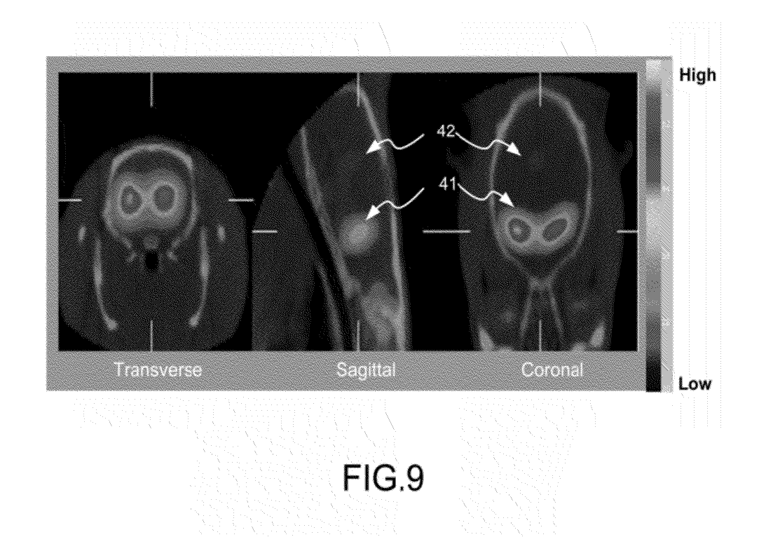

FIG. 9 is a view showing a combined image of a mouse brain obtained through microSPECT and microCT. As shown in the figure, a combined image of a mouse brain is obtained through microSPECT and microCT, where 5 mCi of I-123-Epidepride is injected into the mouse brain. After comparison, obvious absorption is found in striatum [41], thalamus and midbrain [42], which means that I-123-Epidepride can enter an animal's brain to be acted as an imaging agent for dopamine D₂ receptors.

Thus, Epidepride labeled by I-123 can be strongly bonded to the dopamine D₂ receptor with a rare characteristic of non-specificity. The agent can be developed into a tracer for areas other than striatum, like thalamus and temporal cortex, whose densities of D₂ receptors are low. Besides, it is shown in the above imaging process that I-123-Epidepride can enter an animal's brain wih a high affinity and thus is suitable for making a medicine for diagnosing schizophrenia.

To sum up, the present disclosure is a method of labeling dopamine D₂ receptors using a radiosynthesized ligand of iodine/I-123-Epidepride, where Epidepride labeled by I-123 can be strongly bonded to dopamine D₂ receptors with a characteristic of non-specificity and can be developed into a tracer for areas other than striatum and thus can be made into a medicine for diagnosing schizophrenia.

The preferred embodiment herein disclosed is not intended to unnecessarily limit the scope of the disclosure. Therefore, simple modifications or variations belonging to the equivalent of the scope of the claims and the instructions disclosed herein for a patent are all within the scope of the present disclosure.

What is claimed is:

1. A method of labeling dopamine D₂ receptors using a radiosynthesized ligand of iodine(I)-123-epidepride, the method comprising:
obtaining and oscillating a precursor of Sn-Epidepride ((S)-5-(tri-n-butyltin)-N-((1-ethyl-2-pyrrolidinyl)methyl)-2,3-dimethoxybenzamide) added with methanol to obtain a mixed solution of Sn-Epidepride;
mixing said mixed solution of Sn-Epidepride with a solution of I-123-ammonium iodide(NH₄I) and filling and oscillating with a solution of hydrogen peroxide to be stayed still to process destannylation;
filling said mixed solution of Sn-Epidepride with a solution of sodium bisulfite to stop destannylation and, after destannylation, adding said mixed solution of Sn-Epidepride with a saturated buffer solution of disodium hydrogen phosphate to be neutralized;
filling said mixed solution of Sn-Epidepride into a column, washing out un-reacted I-123 ions from said column with sterile water and eluting said column with 100% dehydrated alcohol to obtain a product of I-123-Epidepride having a radiochemistry purity higher than 90%; and
filtering out said product of I-123-Epidepride through a filtering cartridge to be stored in a sterile glass bottle,
wherein, on using said product of I-123-Epidepride, said product is diluted with a sterile saline solution to obtain a final solution having an ethanol density smaller than 20%.

2. The method according to claim 1,
wherein said Sn-Epidepride precursor has an amount of 150˜250 μg.

3. The method according to claim 1,
wherein said methanol has an amount of 50˜150 μg.

4. The method according to claim 1,
wherein said solution of I-123-NH₄I has an amount of 200˜300 μl.

5. The method according to claim 1,
wherein said solution of I-123-NH₄I has an activity of 100˜200 millicuries (mCi).

6. The method according to claim 1,
wherein said I-123-NH₄I is obtained through an irradiation of Xe-123 gas target by a proton beam of compact cyclotron.

7. The method according to claim 1,
wherein said solution of hydrogen peroxide has an amount of 50˜150 μl.

8. The method according to claim 1,
wherein a period of time of said staying still is a period of time of 5˜15 minutes.

9. The method according to claim 1,
wherein said solution of sodium bisulfite has an amount of 250˜350 μl.

10. The method according to claim 1,
wherein said buffer solution of disodium hydrogen phosphate has an amount of 1˜3 ml.

11. The method according to claim 1,
wherein said 100% dehydrated alcohol has an amount of 450˜550 μl.

12. The method according to claim 1,
wherein said filtering cartridge has 0.15˜0.25 μm openings.
